(19) United States
(12) Patent Application Publication
(10) Pub. No.: US 2009/0069702 A1
(43) Pub. Date: Mar. 12, 2009

(54) Method and apparatus for a heart rate indicator fabric

(76) Inventors: See Kee How, Singapore (SG); Mann Weng Yong, Singapore (SG)

Correspondence Address: Nields & Lemack, 176 E. Main Street, Suite #5, Westboro, MA, 01581, US

(21) Appl. No.: 11/900,058

(22) Filed: Sep. 10, 2007

(51) Int. Cl. A61B 5/02

(52) U.S. Cl. 600/508

(57) ABSTRACT

The present invention relates to a method and apparatus for having a fabric that is able to indicate heart rate zones by using colour indicators which comprises of one or a plurality of sensors embedded in the fabric for the detection of heart rate and the signal from the sensor is transmitted to at least a microprocessor whereby the output from the microprocessor will drive at least one or a plurality of light emitting devices and audible signals will be produced. The emitted light is propagated through a medium providing a visual indication to an observer of heart rate zone on the fabric. The audio signal is driven by output signal from the microprocessor to indicate different heart rate zones. Said medium is attached or woven into natural or synthetic yarn to produce light transmitting fabric that reveals different colours. Said sensors, microprocessor and light emitting devices are embedded or attached in the fabric.

### FIELD OF THE INVENTION

The present invention relates to a method and apparatus for having a fabric that is able to indicate heart rate zones by using colour indicators which comprises of one or a plurality of sensors embedded in the fabric for the detection of heart rate and the signal from the sensor is transmitted to at least a microprocessor whereby the output from the microprocessor will drive at least one or a plurality of light emitting devices and audible signals will be produced. The emitted light is propagated through a medium providing a visual indication to an observer of heart rate zone on the fabric. The audio signal is driven by output signal from the microprocessor to indicate different heart rate zones. Said medium is attached or woven into natural or synthetic yarn to produce light transmitting fabric that reveals different colours. Said sensors, microprocessor and light emitting devices are embedded or attached in the fabric.

### PRIOR ART & BACKGROUND OF THE INVENTION

There are devices in the market like the heart rate monitor that are used to accurately detect heart rate. However, such devices require users to put on extra objects on their bodies. A basic heart rate monitor comes in the form of a sensor, a transmitter, a receiver and a processor that will process the transmitted signal and output on a visual display or a sensor and a processor with a display unit. The extra objects attached to the body are cumbersome and not visually appealing.

The current invention utilises the basic mechanisms of heart rate detection but the difference is the said heart rate detector and display unit are interwoven or attached into the fabric. The fabric can be manufactured into apparels or wear for users to wear like a normal wear. The fabric will display different colours for different heart rate zones and audible signals will be produced.

Fabric refers to any material made through weaving, knitting, crocheting or bonding.

The invention can help save lives who may be suffering from cardiac-related diseases as the fabric will change colour and produce sound to show the different heart rate zones so as to make the user aware of their heart rate and take precautionary measures before they collapse. The fabric which when made into apparel can be worn by athletes and can be used as a training tool for athletes to achieve not only performance but also fitness needs. The fabric is also for the visually impaired users as audible signals are produced to indicate the heart rate zones.

### SUMMARY OF THE INVENTION

The present invention relates to a method and apparatus for having a fabric that is able to indicate heart rate zones by using colour indicators which comprises of one or a plurality of sensors embedded in the fabric for the detection of heart rate and the signal from the sensor is transmitted to at least a microprocessor whereby the output from the microprocessor will drive at least one or a plurality of light emitting devices and audible signals will be produced. The emitted light is propagated through a medium providing a visual indication to an observer of heart rate zone on the fabric. The audio signal is driven by output signal from the microprocessor to indicate different heart rate zones. Said medium is attached or woven into natural or synthetic yarn to produce light transmitting fabric that reveals different colours. Said sensors, microprocessor and light emitting devices are embedded or attached in the fabric.

The present invention for having a heart rate indicator fabric consists of the following:
Sensors that pick up bioelectric signals from the surface of the body;
Microprocessor that consists of input port, output port, memory, a processor etc;
Said microprocessor is programmed to read the said bioelectric or biomechanical signals from the sensors and interpret the signals with the preprogrammed settings in the said microprocessor;
Said microprocessor controls the light emitting devices and audio system to reflect the heart rate zones;
Light propagating medium controlled by the said microprocessor that transmits the light from the said light emitting devices;
Audio system that is controlled by the said microprocessor producing audible signals;
Method of having the microprocessor, sensors, light emitting sensors, input console, display system and audio system build on a flexible strip.
Method of having light propagating medium and the said flexible strip interweaved with other yarns to form a fabric;

In the first preferred embodiment, sensors, microprocessor, audio system and light emitting devices are fabricated on a strip. Said strip is preferably flexible and soft to be interwoven with other yarns and medium. The light emitting devices are connected to at least one of the yarns or medium that can propagate light.

Sensors are used to pick up bioelectric events from the surface of the body. As a result of the advantages of electric and electronic methods of measurement, it is common to convert into electrical quantities all non-electrical phenomenon associated with the subject that is measured with the help of a sensor. The sensor converts a physical measured signal from the subject to an electrical signal. The sensor should be minimally invasive and interface with the living system with minimum extraction of energy.

Signal conditioning circuitry is built to process the signals before sending said signal to the microprocessor. Signal conditioners vary in complexity. It usually includes functions such as amplification, filtering, analog to digital conversion and digital to analog conversion.

Said conditioning circuitry consists of filters and amplifiers to amplify the signals and remove noise. It helps in increasing the sensitivity of the said detection by amplification of the original signal.

Said microprocessor is programmed to activate different light emitting devices for different heart rate zones.

Said microprocessor is programmed to activate different tones to the audio system for different heart rate zones.

In the second preferred embodiment, an input console is present which allows user to enter the desired heart rate into the said microprocessor. At least one heart rate value is entered by the user. Alternatively, age can be entered by the user. A formula in the said microprocessor will calculate the different heart rate zones. Said formula is known as the Karnoven formula whereby the maximum heart rate is derived by subtracting the user's age from 220.

Said microprocessor processes the detected signals and interpret readings based on a pre-program heart rate zones set by the user through the input console. Microprocessor will have at least one heart rate zone. At the mentioned zone, the microprocessor will activate at least one light emitting device and activate an audio system by producing a signal to it to indicate the colour and tone respectively. For another heart rate zone, the microprocessor will activate a singular or a plurality of lights emitting devices and output signal to the audio system to produce a different tone. The purpose is to differentiate the different heart rate zones.

In the two embodiments, output signals from the microprocessor are to be conditioned or processed before feeding to the light emitting devices and the audio system.

Said light emitting devices are connected to one or many light propagating mediums to display colours.

Said light propagating medium has light propagating characteristics with high refractive index so that the light rays will be confined in the medium.

Said audio system consists of pre-amplification module and a sound producing device to project audible signals.

### BRIEF DESCRIPTION OF THE DRAWINGS

Illustrative embodiments of the present invention will now be described, by way of example, with reference to the accompanying drawings, in which:

FIG. 1: Representation of the invention in block diagram for the first preferred embodiment

FIG. 2: Representation of the invention in block diagram for the second preferred embodiment

FIG. 3: The sensor, microprocessor, light emitting devices and a speaker fabricated on a flexible medium interweaved with other yarn and light propagating medium to create a fabric

FIG. 4: The sensor, microprocessor, light emitting devices, input console a display and a speaker fabricated on a flexible medium interweaved with other yarn and light propagating medium to create a fabric

FIG. 5: A flow chart of the logic of the microprocessor for the first embodiment

FIG. 6: A flow chart of the logic of the microprocessor for the second embodiment

### DETAILED DESCRIPTION OF THE PREFERRED EMBODIMENTS

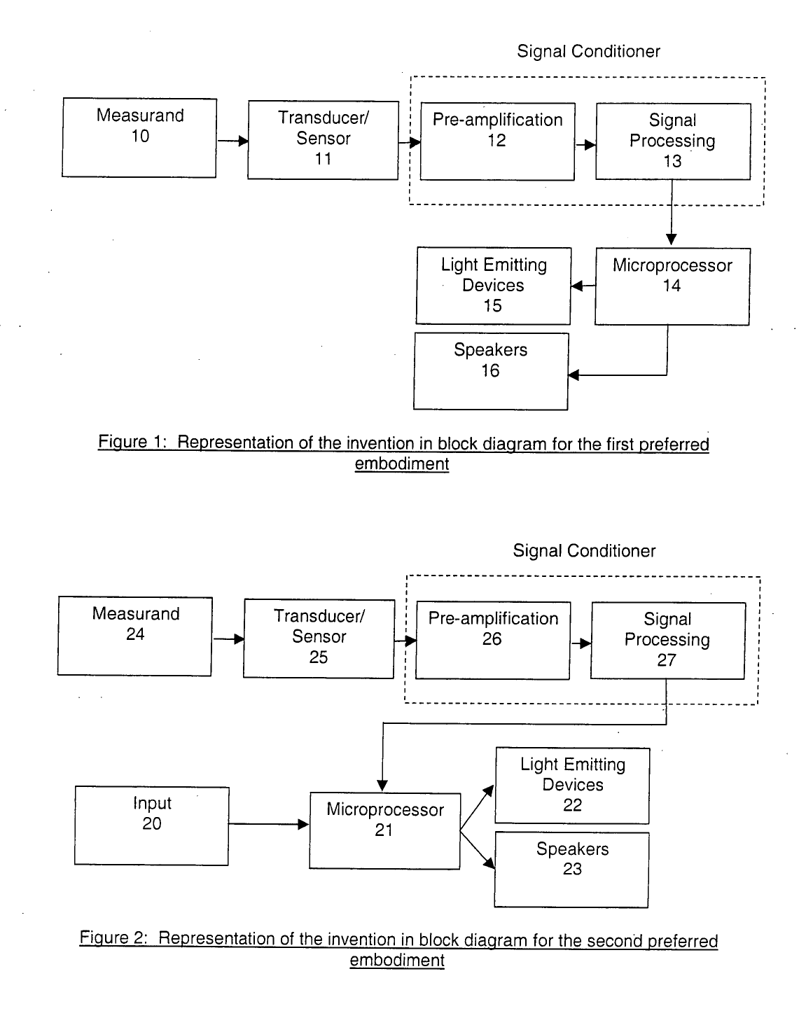

With reference to FIG. 1, said invention can be represented by the block diagram. The measurand (Item 10) is the physical quantity or condition that the instrumentation system measures. The source for the measurand (Item 10) is the living being body which generates a variety of signals. The measurand (Item 10) may be on the surface of the body or may be blood pressure in the chamber of the heart. There are many biomedical signals but of interest pertaining to the invention are the bioelectric signals, biomechanical signals and bio-impedance signals.

Bioelectric signals are generated by many cells and muscle cells. Their basic source is the cell membrane potential which under certain conditions may be excited to generate the action potential. The electric field generated by the action of many cells constitutes the bio-electric signal.

Biomechanical signals originate from some mechanical function of the biological system. They include all types of motion and displacement signals, pressure and flow signals etc.

Bio-impedance signals originate from the impedance of the tissue. The measurement of skin resistance is one example.

The intended measurand (Item 10) determines the transducer or sensor (Item 11) used in the next stage.

The transducer (Item 11) used is to measure heart rate. The primary signal characteristics of a heart rate ranges from 25 to 300 beats per minute. The normal living being heart rate at rest is from 60 to 90 beats per minute. The transducer (Item 11) ideal for the signal characteristics can be derived from ECG (electro cardiogram) or arterial blood pressure waveform or photoelectric plethysmograph.

Piezo-electric transducer or displacement sensor (Item 11) works on the principle that when an asymmetrical crystal lattice is distorted, a charge re-orientation takes place, causing a relative displacement of negative and positive charges. The displaced internal charges induce surface charges of opposite polarity on opposite sides of the crystal. Surface charge can be determined by measuring the difference in voltage between electrodes attached to the surfaces of the measurand (Item 10).

Piezo-electric materials are available as polymeric films such as polyvinylidene fluoride (PVDF). These films are thin, light weight and flexible and can be easily cut for adaptation to uneven services. There is quartz which offers high mechanical and thermal stability and has volume resistivity higher than 10⁴Ω per centimeter and small internal electric loss.

Therefore if the fabric is weaved with such a transducer/sensor (Item 11), the living being body slight movement would create a pressure that would trigger the said sensor (Item 11) to register some bioelectric events. A combination of sensors may be incorporated to detect the heart pulse.

In order for the signal from the sensor (Item 11) to be compatible with the microprocessor (Item 14), it is common to normalize the electrical signals produced by the said sensor (Item 11) by the signal conditioner.

The signals available from the transducers/sensors (Item 11) are often very small in magnitude usually at microvolt levels. The pre-amplifier (Item 12) boosts the level of input signal to match the requirements of the microprocessor (Item 14) after signal processing (Item 13).

The signal processing (Item 13) performs operation like filtering unwanted frequency signals, converting analog to digital signal. The digitized signal is fed to the microprocessor (Item 14) for processing.

At the microprocessor (Item 14), the processed signal would appear as a series of pulses. Said microprocessor (Item 14) is programmed to read the pulse width and interval between each pulse to determine the heart rate. Once the heart rate is determined, the respective light emitting devices (Item 15) and audible signals (Item 16) will be activated to indicate the different heart rate zones through visual and audio signals.

Light emitting devices (Item 15) are connected to light propagating yarns that made up the fabric. The light propagating yarns are interwoven with other natural or synthetic yarns to form the fabric. Said light emitting devices could be light emitting diodes. Said light propagating yarns could be optical fibers. The light propagating yarns reveal the different colours with the changing heart rate.

Miniature speakers (Item 16) are installed on the fabric to allow signals from the microprocessor (Item 14) to be audible. The output audio signal can be a beep to inform the user a zone has been reached or the output audio signal can be series of beeps synchronize with the pulse rate.

With reference to FIG. 2, is another representation of the second embodiment whereby an input module (Item 20) allows user to enter the desired heart rate and age.

The entered value is stored into the microprocessor's (Item 21) memory for comparison with the measured value by the sensor (Item 25)

The microprocessor (Item 21) takes the input from the user and calculates the different heart rate zones namely resting, aerobic, high aerobic and anaerobic zones by using the Karnoven formula.

If the user's heart rate is at a certain level, the light emitting devices and the speakers will be activated. Preferably, the speakers produce a beep for a short time to inform the heart rate zone has been reached. Alternatively, the output audio signal can be series of beeps synchronize with the pulse rate.

The light from the light emitting devices are lit to reflect the different heart rate zones. For example, yellow for resting, orange for aerobic, red for higher aerobic and bright red for anaerobic.

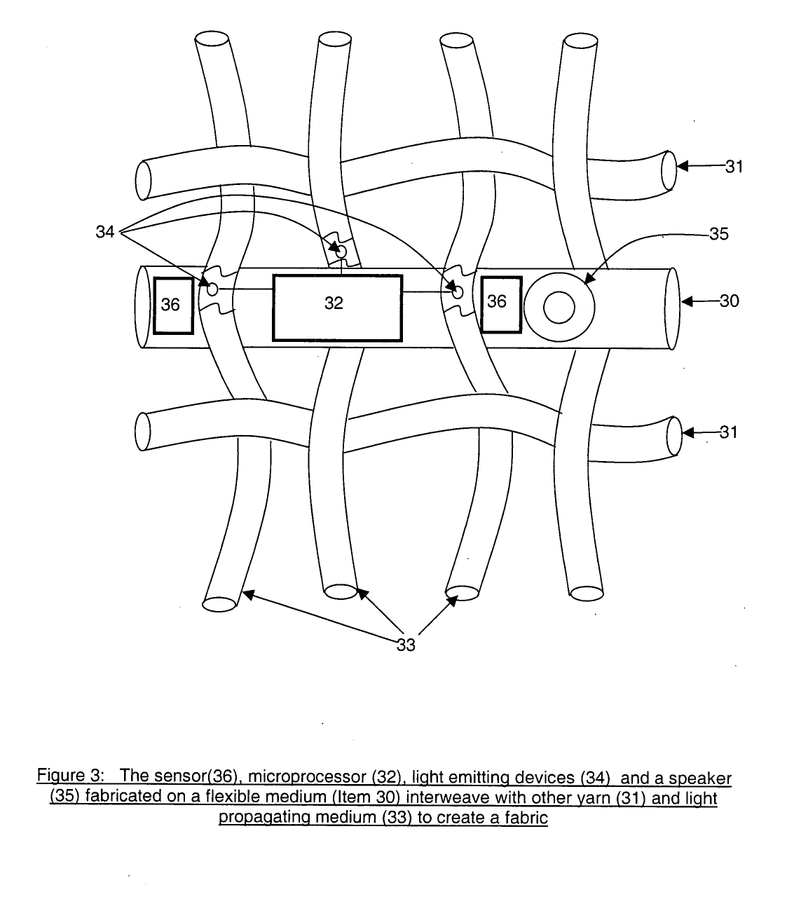

With reference to FIG. 3, the first preferred embodiment whereby the microprocessor (Item 32), speaker (Item 35), light emitting devices (Item 34) are fabricated on a strip of flexible material is interwoven with other yarns (item 31) and light propagation medium (Item 33). Said yarns may be natural or synthetic.

Said strip (Item 30) is of flexible and waterproof construction.

The electrodes or sensors (Item 36) and the microprocessor (Item 32) are embedded into the said strip (Item 30) with the electrical connection to the light emitting devices (Item 34).

Said light emitting devices are electrically connected to the light propagating medium (Item 33).

Said electrical connection is also connected from the microprocessor (Item 32) to the speaker (Item 35) via amplification circuit.

Within the said strip, stored energy supplies energy to the microprocessor (Item 32), the speaker (Item 35), the light emitting devices (Item 34) and other electronics.

Said stored energy may be from a battery or electrochemical cells capable of producing an electromotive force chemically.

Said strip can be fabricated as solar panel and the light energy is converted into electrical charges and the charges are stored and used to energize the said electronics.

The light propagating medium (Item 33) are preferably made of clear, translucent medium so that the different colours can propagate and visually visible by the living being eye.

Preferably the medium (Item 33) is optical fiber which is made of plastic also known as plastic optical fiber (POF).

The terminations of the said medium (Item 33) are to be placed with light emitting devices (Item 34). Said light emitting devices can be light emitting diodes (LEDs).

Once the sensors (Item 36) detect a pulse rate, the microprocessor (Item 32) will compare the measured pulse rate with an internal table programmed in it. The matched pulse rate with the table will trigger the appropriate light devices (Item 32) and right tone to the speaker (Item 35). For instance, if the resting heart rate is below 100, the light and speaker will not be lit and turned on respectively. If a pulse rate of 100 is detected, at least one light device will be on with a colour assigned to it. If a pulse rate of more than 129 is detected, a second colour will be lit and the first lit light device can be either on or off. The light devices (Item 32) are connected to the terminations of the light propagating medium (Item 33) so that the lights are confined in the said medium.

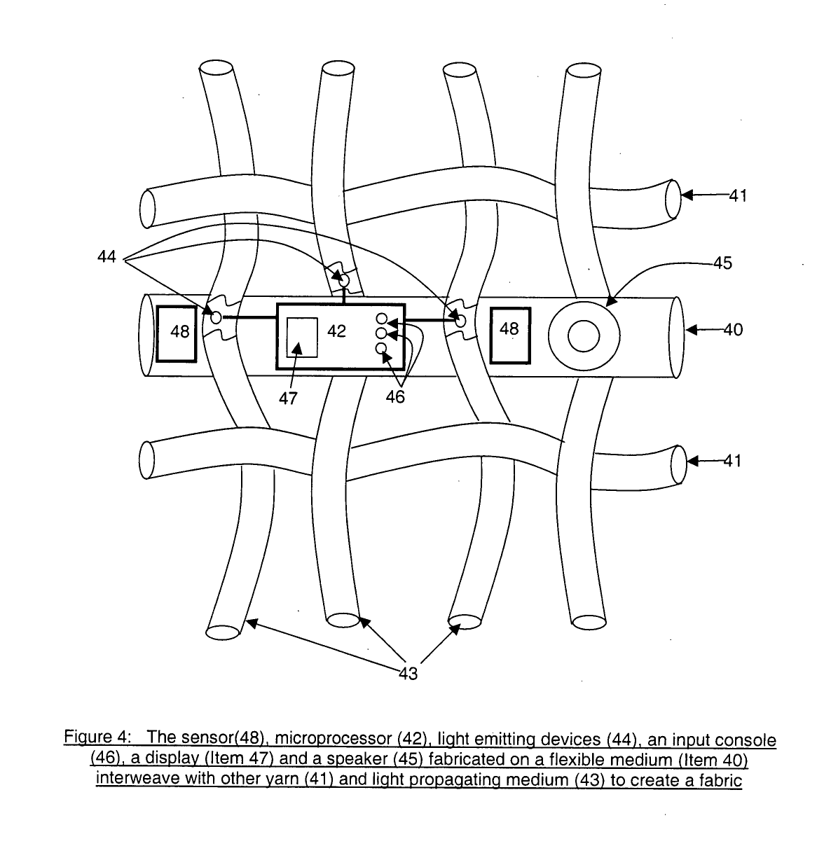

With reference to FIG. 4, a second preferred embodiment is presented.

The sensor (Item 48), microprocessor (Item 42), speaker (Item 45), the display (Item 47) and the input console (Item 46) are fabricated on a flexible medium to be interwoven with other yarns or fibers.

The input console (Item 46) comprises of buttons that can be entered by the user to set their heart rate zones.

The input values can be observed through a display (Item 47) which is a preferably a liquid crystal display.

Instead of a pre-programmed heart rate zones as in the first embodiment, the current embodiment allows user to enter one self's desired heart rate.

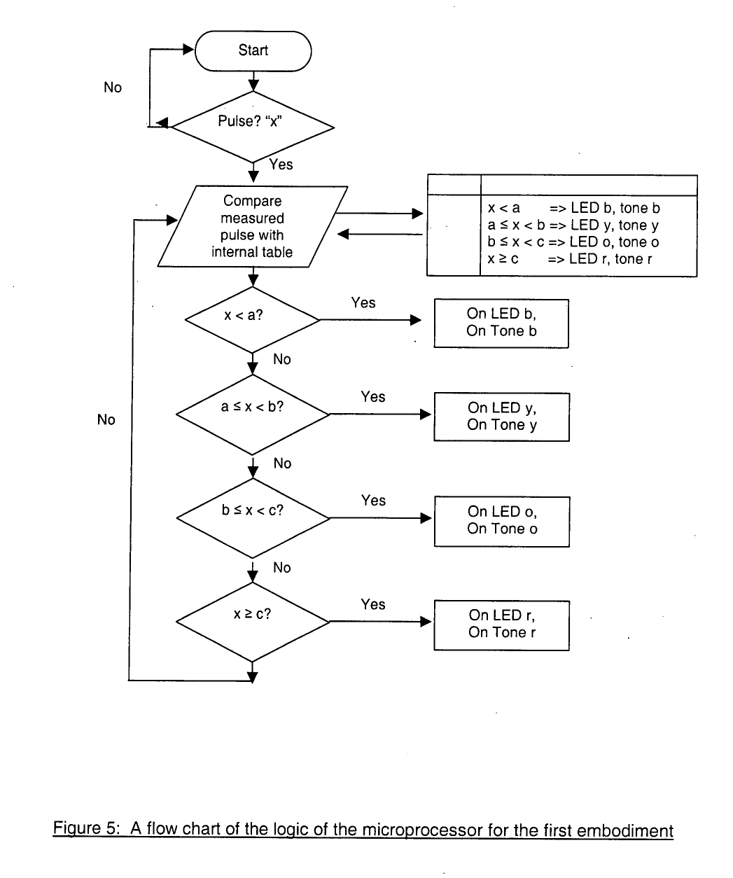

With reference to FIG. 5, a flow chart of the sequence of the first preferred embodiment is presented.

Immediately the fabric with the sensor detected a pulse rate, x, the microprocessor will compare the measured pulse rate, x with the internal table whereby the values a, b and c are pre-set.

If the pulse, x is less than the preset value, a, the light emitting diode b and a tone b will be activated.

If the pulse, x is more than or equal the preset value, a but less than preset value, b, light emitting device y and tone y will be activated.

If the pulse, x is more than or equal the preset value, b but less than preset value, c, light emitting device o and tone o will be activated.

If the pulse, x is more than the preset value, c, the light emitting diode r and a tone r will be activated.

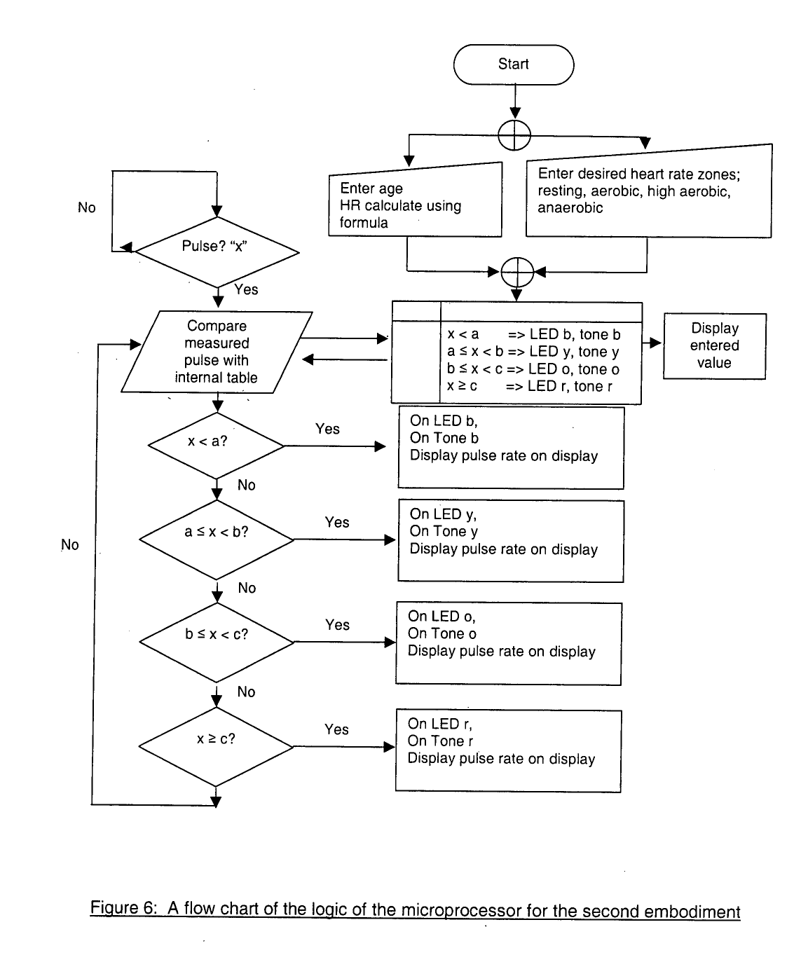

With reference to FIG. 6, a flow chart of the sequence of the second preferred embodiment is presented.

In order to use the said heart rate monitoring fabric, user has to enter one self's desired heart rate. Alternatively, user can enter the age only and let the microprocessor calculate the different heart rate zones by using the Karnoven formula. Said formula calculate the maximum heart rate using the formula 220−user's age.

Either method of entering the values, the values will be stored in the memory of the microprocessor. The measured value is then compared with the table and the detected pulse rate will correspond with the light emitting diode and the tone.

The measured pulse rate can be observed on the display.

The entered value by the user can be observed on the display.

Modifications within the spirit and scope of the invention may readily be effected by persons skilled in the art. It is to be understood, therefore, that this invention is not limited to the particular embodiments described by way of example hereinabove.

1. A fabric adapted to visually indicate heart rate zones of a living being comprising:
a power source;
a sensor embedded in said fabric adapted to detect the heart rate of said living being;
a microprocessor in communication with said sensor to process the detected heart rate to determine said heart rate zones, wherein said microprocessor comprises an output, and said output from the microprocessor is in communication with a light emitting device;
a medium that allows the light emitted from said light emitting device to propagate, thereby providing a visual indication on said fabric of said heart rate zone; and
natural or synthetic yarns or fibers with which said medium is interwoven or attached.

2. The fabric of claim 1 further comprising an audio device, wherein said microprocessor comprises a second output, and said second output is in communication with said audio device.

3. The fabric of claim 1 further comprising an input console and a display system in communication with said microprocessor.

4. The fabric of claim 1, further comprising a flexible strip, wherein said electrical power source, said sensor, and said microprocessor are affixed to said flexible strip.

5. The fabric of claim 4, wherein said electrical power source is enclosed in said flexible strip.

6. The fabric of claim 4 wherein said light emitting device is in communication with said flexible strip via at least one connecting conductor.

7. The fabric of claim 3 further comprising a flexible strip, wherein said electrical power source, said sensor, said display system and said microprocessor are affixed to said flexible strip.

8. The fabric of claim 2 further comprising a flexible strip, wherein said electrical power source, said sensor, said audio device and said microprocessor are affixed to said flexible strip.

9. The fabric of claim 7 wherein said input console is affixed to said flexible strip.

10. The fabric of claim 4 wherein said microprocessor is enclosed in said flexible strip.

11. The fabric of claim 4 wherein said sensors are affixed on the periphery of said flexible strip.

12. The fabric of claim 4 wherein said medium has at least one end and said light emitting device is in communication with said end of said medium to enable the light emitted by said light emitting device to propagate in said medium.

13. The fabric of claim 1, further comprising a plurality of light emitting devices, each of said devices emitting a different color light.

14. The fabric of claim 13, wherein said microprocessor comprises a storage element having at least one threshold value, wherein the light color activated by said microprocessor is related to said detected heart rate of said living being and said threshold value.

15. The fabric of claim 14, further comprising an input console, wherein said threshold is entered via said input console.

16. The fabric of claim 2, wherein said microprocessor comprises a storage element having at least one threshold value, wherein the audio tone activated by said microprocessor is related to said detected heart rate of said living being and said threshold value.

17. A method of visually indicating heart rate zones of a living being comprising:
Providing a power source; a fabric; a sensor embedded in said fabric adapted to detect the heart rate of said living being; a microprocessor in communication with said sensor to process the detected heart rate to determine said heart rate zones, wherein said microprocessor comprises an output, and said output from the microprocessor is in communication with a light emitting device; a medium that allows the light emitted from said light emitting device to propagate, thereby providing a visual indication on said fabric of said heart rate zone;
Interweaving said medium with natural or synthetic yarns or fibers;
Detecting said living beings heart rate with said sensor;
Providing said detected heart rate to said microprocessor; and
Illuminating said light emitting device by said microprocessor in response to said detected heart rate.

18. The method of claim 17, wherein there is a plurality of light emitting devices and wherein said microprocessor is in communication with said plurality of light emitting devices, each of said light emitting devices is adapted to emit different light color.

19. The method of claim 18, wherein said microprocessor comprises a storage element having at least one threshold value, and said microprocessor illuminates a different light color based on said detected heart rate and said threshold value.

20. The method of claim 19, wherein said microprocessor is in communication with an input console and said method further comprises entering, via said input console, said threshold value.

21. The method of claim 19, wherein there is an audio device capable of emitting a plurality of audible tones, and wherein said microprocessor is in communication with said audio device, said method further comprising emitting one of said plurality of audible tones through said audio device, based on said threshold value and said detected heart rate.
